(19) United States
(12) Patent Application Publication
(10) Pub. No.: US 2006/0269498 A1
(43) Pub. Date: Nov. 30, 2006

(54) Hair shaping composition comprising at least one polyguanidine other than hydroxide

(76) Inventors: Gerard Malle, Villiers S/Morin (FR); Christian Blaise, Saint Mande (FR); Xavier Radisson, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/434,741

(22) Filed: May 17, 2006

Related U.S. Application Data
(60) Provisional application No. 60/685,865, filed on Jun. 1, 2005.

(30) Foreign Application Priority Data
May 17, 2005 (FR) 05 51276

(51) Int. Cl. A61K 8/00

(52) U.S. Cl. 424/70.2

(57) ABSTRACT

Disclosed herein is a ready-to-use cosmetic composition for shaping keratin fibers, comprising, in a cosmetically acceptable medium, as active agent for shaping, a polyguanidine not belonging to the hydroxide family. Also disclosed herein is a kit comprising at least two compartments to be brought into contact to form the ready-to-use composition. Further disclosed herein is a method for shaping keratin fibers comprising applying this composition to the keratin fibers.

This application claims benefit of U.S. Provisional Application No. 60/685,865, filed Jun. 1, 2005, the contents of which are incorporated herein by reference. This application also claims benefit of priority under 35 U.S.C. § 119 to French Patent Application No. FR 05 51276, filed May 17, 2005, the contents of which are also incorporated herein by reference.

Disclosed herein is a ready-to-use cosmetic composition for shaping keratin fibers, comprising, as the active shaping agent, at least one polyguanidine not belonging to the hydroxide family. Also disclosed herein is a kit comprising at least two compartments that are to be brought into contact to form a ready-to-use composition. Further disclosed herein is a method for shaping keratin fibers comprising applying said composition to the keratin fibers.

As used herein, the term “keratin fibers” or “keratinous materials” is understood to mean fibers of human or animal origin, such as hair, bristles, eyelashes, wool, angora, cashmere, and fur. In at least one embodiment, the keratin fibers may be hair.

As used herein, the term “permanent shaping” encompasses curling, waving, perming, and/or setting of the hair, for instance, Caucasian, Asiatic, and North African hair.

As used herein, the term “polyguanidine not belonging to the hydroxide family” is understood to mean any organic compound whose formula comprises the following group at least twice:
a carbon atom joined by a double bond to another nitrogen atom and by a single bond to two other nitrogen atoms and not comprising hydroxide ions in its chemical formula. For instance, this term may encompass polyguanidines and their organic and inorganic salts not containing hydroxide ions in their chemical formulas.

In at least one embodiment of the present disclosure, the at least one polyguanidine may contain the guanidine group at least twice, for example, 2 or 3 times.

Hereinafter, the expression “ranging from x to y” means in the range from x to y, the limits x and y being included.

Conventionally, two techniques may be employed to obtain permanent deformation of the hair. They are based on rupture of the disulphide bonds that are present in keratin (cystine):
The first technique comprises effecting the opening of the disulphide bonds using a composition comprising a reducing agent, then, after optionally rinsing the hair, reconstituting the disulphide bonds by applying to the hair, previously stretched by rollers and/or other devices and/or shaped and/or smoothed by other means, an oxidizing composition (also called a setting lotion), in order to give the hair the desired shape. This technique may be used for waving, shaping, straightening, and/or smoothing the hair.
The second technique comprises carrying out an operation called lanthionization, using a composition comprising a base belonging to the hydroxide family. This technique leads to replacement of the disulphide bonds (—CH₂—S—S—CH₂—) with lanthionine bonds (—CH₂—S—CH₂—). This lanthionization operation involves two consecutive chemical reactions:
The first reaction comprises beta-elimination on the cystine brought about by a hydroxide ion, leading to rupture of this bond and formation of dehydroalanine, as shown, for example, in reaction scheme (A) below. 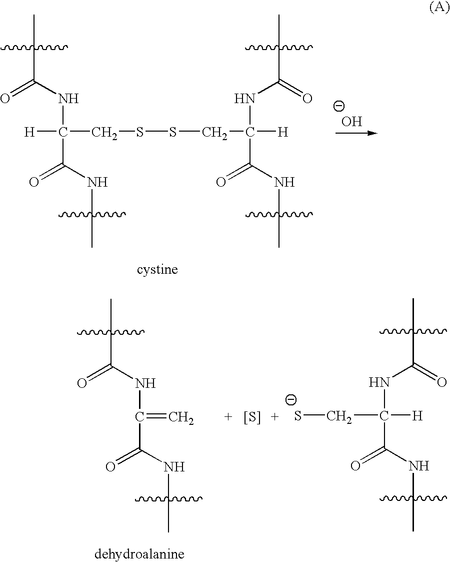
The second reaction is a reaction of the dehydroalanine with a thiol group, as shown, for example, in reaction scheme (B) below. The double bond of the dehydroalanine formed is a reactive double bond, which is able to react with the thiol group of the cysteine residue that was released to form a new bond called a bridge or linkage or lanthionine residue.

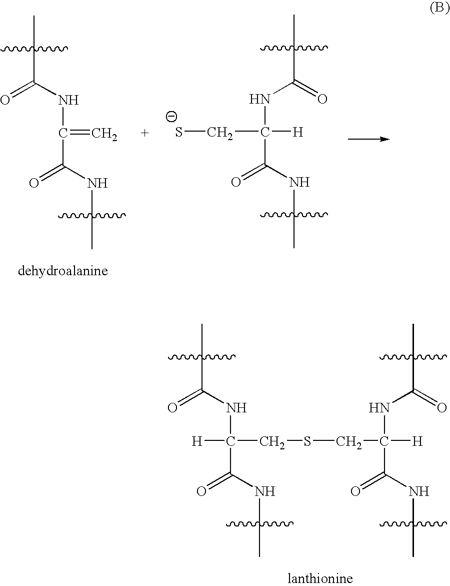

Compared with the first technique employing a reducing agent, this lanthionization technique does not require a setting stage, since formation of the lanthionine bridges is irreversible. It is therefore effected in a single stage and may be used for waving, shaping, straightening, and/or smoothing the hair. Typically, this technique is used for shaping naturally frizzy hair.

For the first technique, the reducing compositions generally used for the first stage of a perming and/or setting operation may comprise at least one reducing agent chosen from thiols, sulphites, and bisulphites. These agents are generally employed in an essentially aqueous medium at concentrations ranging from 0.5 to 1 M to effect opening of the disulphide bonds. Examples of thiols include, but are not limited to, thioglycolic acid, cysteamine, glycerol monothioglycolate, thiolactic acid, and cysteine. Thioglycolic acid may be effective for reducing the disulphide bonds of keratin at an alkaline pH, for example, in the form of ammonium thioglycolate, and is the product most commonly used for hair waving. However, thioglycolic acid should be used in a sufficiently basic environment (for example, at a pH ranging from 8.5 to 9.5) to obtain a satisfactory degree of curling. The use of a thiol at an alkaline pH may lead to degradation of the fiber, for example, alteration of artificial coloring. In addition, thioglycolc acid releases an unpleasant odor, making it necessary to use perfumes that are more or less effective at masking the odor.

Sulphites or bisulphites are conventionally used for shaping the hair. However, these compounds are less effective than thiols and have disadvantages similar to thiols.

Thiols and sulphites (or bisulphites) also have the drawback of poor stability in aqueous solution.

Generally speaking, the durability of the deformation effects obtained with thiols and sulphites by reduction of the disulphides is inferior to that obtained by the lanthionization technique.

For the second technique, the compositions generally used for carrying out lanthionization comprise, as a base, a hydroxide such as sodium hydroxide, guanidinium hydroxide, or lithium hydroxide. These active agents of lanthionization, permitting opening of the disulphide bonds by a beta-elimination mechanism, are generally used in a water-oil emulsion at molar concentrations ranging from 0.4 to 0.6 M, and left to act for a period of time ranging from 10 to 15 minutes at room temperature. Sodium hydroxide is the agent used most commonly. Guanidinium hydroxide is also frequently used. These two hydroxides, of sodium and of guanidinium, are generally used for shaping and/or straightening naturally frizzy hair. These compounds may have several advantages over ammonium thioglycolate and sulphites, for example, the absence of unpleasant odor, a single working stage (i.e., shorter treatment time), and greater durability and effectiveness of deformation of the hair.

However, these hydroxides can have the drawback of being caustic. This causticity affects the scalp, causing irritation that may be severe. A partial remedy comprises prior application of an oily protective cream on the scalp, often called “base” or “cream base,” where the term “base” is not used here in its meaning of basic agent in the chemical sense. When the protective cream is combined with the hydroxide in a single composition, the latter is generally called “no-base,” as the opposite of the above designation. This “no-base” technology is frequently employed.

The causticity of the hydroxides may also affect the condition of the hair, making it rough to the touch and/or more brittle, and this brittleness can extend to disintegration, breaking, and/or dissolving of the hair if the treatment is prolonged. In certain cases, the hydroxides may also cause decolorization of the hair's natural color.

The formulas comprising sodium hydroxide are generally called “lye relaxers,” and those not comprising sodium hydroxide are called “no-lye relaxers.”

Typical “no-lye” straightening formulas may employ guanidinium hydroxide. As guanidinium hydroxide is unstable, it is generated at the time of use by mixing guanidine carbonate and a source of hydroxide of very low solubility, such as calcium hydroxide. The reaction between these two compounds leads to the formation of guanidinium hydroxide and calcium carbonate, which is precipitated in the composition. The presence of this precipitate can make final rinsing of the hair more difficult and can leave mineral particles on the hair and the scalp, giving them a rough feel and an unsightly appearance resembling dandruff. The advantages of guanidinium hydroxide (“no-lye”) over sodium hydroxide (“lye”) can include better effectiveness in shaping and/or better skin tolerance. However, these technologies employing bases from the hydroxide family are still very aggressive to the hair and the scalp, and require strict control of the duration of application to avoid excessive irritation and deterioration of the hair, and even breakage. The aggressiveness of the compositions used for lanthionization of the hair, due to the causticity of the hydroxides, is why these compositions are generally not used for hair waving, but are reserved for shaping (hair straightening and/or hair relaxing).

Moreover, hydroxides are known to be good agents for hydrolysis of amide functional groups (see, for example, March's Advanced Organic Chemistry, 5th edition, Wiley Interscience, New York, “Hydrolysis of Amides,” pp. 474 ff.), which may therefore lead to rupture of the peptide bonds by direct nucleophilic attack. It is thus probable that the changes observed in the hair and keratin materials in the broad sense are largely due to partial hydrolysis of the amide bonds of the keratin.

Therefore it is desirable to develop compositions that are less aggressive to the hair and the skin.

Several studies have been conducted for the purpose of overcoming the drawbacks of reducing agents (first technique) and/or of hydroxides (second technique).

Thus, numerous reducing agents have been proposed to replace thioglycolic acid, but thioglycolic acid—in the form of ammonium thioglycolate—is still considered to be the reference compound and the compound that is used most widely in cosmetic formulations, for straightening, shaping, and/or smoothing the hair.

However, the use of reducing agents may lead to insufficient durability for permanent shaping, necessitating frequent repetition of the treatment and leading inevitably to degradation with each extra application. Furthermore, the causticity of the hydroxides generally limits their use to the area of straightening. No study has proposed a base as the active agent for permanent shaping of the hair, which is both effective and less aggressive than soda and hydroxides in the broad sense.

The present inventors have found, surprisingly, that the first stage of the lanthionization process may be effected with polyguanidines that do not belong to the hydroxide family. Excellent results may be obtained in this way in terms of the degree of permanent shaping, and the cosmetic and mechanical characteristics of the hair, with better durability than that obtained with the known reference products, for instance, ammonium thioglycolate, for permanent shaping.

Disclosed herein is a novel cosmetic composition comprising, in a cosmetically acceptable medium, at least one polyguanidine not belonging to the hydroxide family, the cosmetically acceptable medium and the at least one polyguanidine being chosen such that the at least one polyguanidine not belonging to the hydroxide family reacts on the cystines of the keratin fibers, by a beta-elimination reaction, producing dehydroalanine and leading to the formation of lanthionine, to form curls in the keratin fibers with a diameter ranging from 0.2 to 3 cm, in a period of time of less than 60 minutes.

In at least one embodiment, the period of time may be less than 40 minutes, for example, less than 30 minutes.

Polyguanidines suitable for use in accordance with the present disclosure may be chosen, for example, from compounds of formula (I): 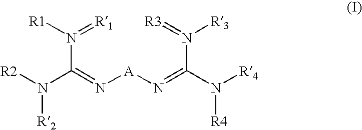
wherein:
R₁, R′₁, R₂, R′₂, R₃, R′₃, R₄, and R′₄, which may be identical or different, are radicals chosen from:
   hydrogen, and
   C₁-C₆ alkyl groups, which may be saturated or unsaturated, linear (for example, methyl and ethyl), branched (for example, isopropyl and tert-butyl), or cyclic (for example, cyclopentyl and cyclohexyl), said alkyl groups being optionally substituted by a radical of formula (II): 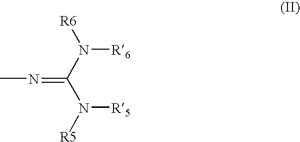
wherein R₅, R′₅, R₆, and R′₆, which may be identical or different, are radicals chosen from:
   hydrogen, and
   C₁-C₆ alkyl groups, which may be saturated or unsaturated, linear (for example, methyl and ethyl), branched (for example, isopropyl and tert-butyl), or cyclic (for example, cyclopentyl and cyclohexyl);
R₁ and R₂ and/or R₃ and R4 may optionally form together a divalent radical chosen from (CH₂)₂, (CH₂)₃, and CH═CH; and
A is chosen from C₂-C₁₂ divalent hydrocarbon radicals, which may be linear or cyclic, saturated or unsaturated, and which may optionally be interrupted by at least one entity chosen from:
   imino, carboxamido, sulphoxide, and sulphone groups, and/or
   at least one heteroatom chosen from sulphur, oxygen, nitrogen, and silicon, and optionally substituted by:
   a linear or branched C₁-C₄ alkyl radical, optionally interrupted by at least one heteroatom as defined above and optionally substituted by a radical of formula (II): 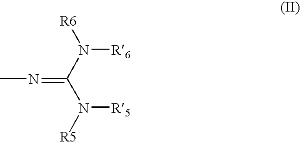
wherein R₅, R′₅, R₆, and R′₆ are defined above.

The compounds of formula (I) may be prepared according to the following synthesis schemes:

Vilsmeyer salt 1 (prepared, for example, by the action of phosgene or of a substitute such as oxalyl chloride or phosphorus oxychloride on tetraalkyl urea) is reacted with diamine 2 (or triamine 3) in a polar aprotic solvent such as acetonitrile in the presence of a tertiary organic base, such as triethylamine to give the compounds of formula (I).

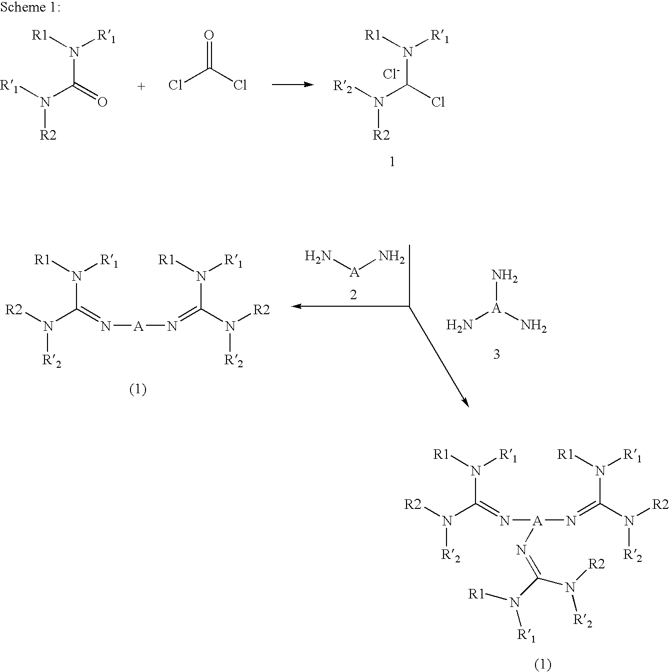

A dihalogenated derivative 4 is reacted with a large excess of tetraalkylguanidine 5 at a temperature close to 100° C. The excess tetraalkylguanidine is removed by evaporation under reduced pressure and the residue is treated with a base, such as sodium ethoxide in ethanol. The salt is removed by filtration then the required derivative of formula (I) is obtained and purified generally by distillation under reduced pressure.

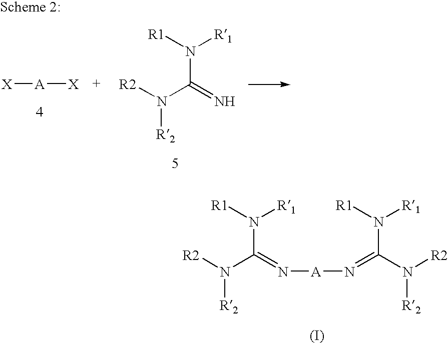

Useful procedures are also described, for example, in the following references:
1) Z.anorg. Allug. Chem. 626: 1583-1590 (2000),
2) Inorg. Chem. 40: 6964-6971 (2001),
3) J. Org. Chem. 68: 8790-8797 (2003), and
4) J. Chem. Soc.; Dalton Trans., 3473-3479 (2000).

In at least one embodiment, the compounds of formula (I) may be chosen, for example, from:
N,N′-bis(1,3-dimethyl-2-imidazolidinylidene)-1,2-benzenediamine (RN: 774610-65-0),
N-[bis(dimethylamino)methylene]-3-(dimethylamino)-5-imino-2-methyl-2,4,6,19-tetraazaeicosan-20-imidamide (RN: 791543-84-5),
N,N′-bis(tetrahydro-1,3-dimethyl-2(1H)-pyrimidinylidene)-1,3-propanediamine (RN: 752232-69-2),
1,1′,1″,1″′-[1-propanediylbis(nitrilomethanetetrayl)]tetrakis-piperidine (RN: 752232-68-1),
poly(oxy-1,2-ethanediyl), α-[2-[(1,3-dimethyl-2-imidazolidinylidene)amino]ethyl]-ω-[2-[(1,3-dimethyl-2imidazolidinylidene)amino]ethoxy] (RN: 742679-23-8),
N″,N″″′-1,2-ethanediylbis[N,N,N′,N′-tetrakis(1-methylethyl)-guanidine (RN: 676488-06-5),
N,N′-bis(1,3-dihydro-1,3-dimethyl-2H-imidazol-2-ylidene)-1,8-naphthalenediamine (RN: 634192-99-7),
N,N′-bis(1,3-dimethyl-2-imidazolidinylidene)-1,8-naphthalenediamine (RN: 501931-38-0),
N″,N″″′-1,8-biphenylenediylbis[N,N,N′,N′-tetramethyl-guanidine] (RN: 495408-66-7),
N″,N″″′-benzo[c]phenanthrene-1,12-diylbis[N,N,N′,N′tetramethyl-guanidine] (RN: 495408-58-7),
N″,N″″′-4,5-phenanthrenediylbis[N,N,N′,N′-tetramethyl-guanidine] (RN: 495408-50-9),
N″,N″″′-(9,10-dihydro-4,5-phenanthrenediyl)bis[N,N,N′,N′-tetramethyl-guanidine] (RN: 495408-44-1),
N″,N″″′-1,2-phenylenebis[N,N,N′,N′-tetramethyl-guanidine] (RN: 495408-35-0),
N″,N″″′-1,8-naphthalenediylbis[N,N,N′,N′-tetramethyl-guanidine]hydrochloride (RN: 443892-20-4),
N″,N″″′-9H-fluorene-4,5-diylbis[N,N,N′,N′-tetramethyl-guanidine] (RN: 443892-12-4),
N′-(tetrahydro-1,3-dimethyl-2(1H)-pyrimidinylidene)-N,N-bis[2-[(tetrahydro-1,3-dimethyl-2(1H)-pyrimidinylidene)amino]ethyl]-1,2-ethanediamine (RN: 395640-62-7),
N″,N″″′,N″″″″-(nitrilotri-2,1-ethanediyl)tris[N,N,N′,N′-tetramethyl-guanidine] (RN: 368866-05-1),
N″,N″″′-[2-[[[bis(dimethylamino)methylene]amino]methyl]-2-methyl-1,3-propanediyl]bis[N,N,N′,N′-tetramethyl-guanidine] (RN: 289474-30-2),
N″,N″″′-(2,2-dimethyl-1,3-propanediyl)bis[N,N,N′,N′-tetramethyl-guanidine] (RN: 289474-29-9),
N″,N″″′-1,3-propanediylbis[N,N,N′,N′-tetramethyl-guanidine](RN:289474-28-8),
N″,N″″′-1,2-ethanediylbis[N,N,N′,N′-tetramethyl-guanidine] (RN: 289474-25-5),
N,N′-bis(1,3-dihydro-1,3-dimethyl-2H-imidazol-2-ylidene)-1,2-ethanediamine (RN: 216873-26-6)
N,N″′-1,2-ethanediylbis[N′N′N″,N″-diethyl-guanidine] (RN: 211869-99-7),
N″,N″″′-[(methylimino)di-3,1-propanediyl]bis[N,N,N′,N′-tetramethyl-guanidine] (RN: 196405-86-4),
N″,N″″′-[oxybis(2,1-ethanediyloxy-3,1-propanediyl)]bis[N,N,N′,N′-tetramethyl-guanidine] (RN: 190442-53-6),
N″,N″″′-[[1,1,3,3-tetramethyl-1,3-disiloxanediyl]di-3,1propanediyl]bis[N,N,N′,N′-tetramethylguanidine] (RN: 175989-14-7),
N″,N″″′-[[1,3-dimethyl-1,3-bis[(trimethylsilyl)oxy]-1,3-disiloxanediyl]di-3,1-propanediyl]bis[N,N,N′,N′-tetramethyl-guanidine] (RN: 172283-48-6),
N,N″-1,6-hexanediylbis[N′-[bis(dimethylamino)methylene urea (RN: 157362-45-3),
[bis(dimethylamino)methylene][3[[[[[bis(dimethylamino)-methylene]amino]carbonyl]amino]methyl]-3,5,5-trimethylcyclohexyl]-urea(RN: 157362-44-2),
N″,N″″′-[1,1′-biphenyl]-2,2′-diylbis[N,N,N′,N′-tetramethyl-guanidine](RN: 144576-63-6),
4,4′-sulphonylbis[N-(1,3-dimethyl-2-imidazolidinylidene)-benzeneamine(RN: 129346-76-5),
N,N′-bis(1,3-dimethyl-2-imidazolidinylidene),2,8-dibenzothiophenediamine-5,5-dioxide (RN: 128169-35-7),
N,N′-bis(1,3-dimethyl-2-imidazolidinylidene)-4,6-dibenzothiophenediamine-5,5-dioxide (RN: 127330-56-7),
N,N′-bis(1,3-dimethyl-2-imidazolidinylidene)-1,2-ethanediamine (RN: 126620-51-7),
N″,N″″′-1,3-phenylenebis[N,N,N′,N′-tetramethyl-guanidine] (RN: 121648-84-8),
N,N′-bis[bis(dimethylamino)methylene]-butanediamine (RN: 114491-72-4),
N″,N″″′-1,4-phenylenebis[N,N,N′,N′-tetramethyl-guanidine] (RN: 113551-45-4),
N″,N″″′-[[1,3-dimethyl-1-[(pentamethyldisiloxanyl)oxy]-3-[(trimethylsilyl)oxy]-1,3-disiloxanediyl]di-3,1-propanediyl]bis[N,N,N′,N′-tetramethyl-guanidine] (RN: 109956-31-2),
N,N″-bis[(dimethylamino)(dipentylamino)methylene]-1,4piperazinedicarboximidamide (RN: 97983-93-2),
N,N″-bis[(dimethylamino)(hexylpropylamino)methylene]-1,4piperazinedicarboximidamide (RN: 97983-92-1),
N,N″-bis[(hexylmethylamino)(methylpropylamino)methylene]-1,4-piperazinedicarboximidamide (RN: 97983-91-0),
N,N″-bis[(butylmethylamino)(hexylmethylamino)methylene]-1,4-piperazinedicarboximidamide (RN: 97983-90-9),
N,N″-bis[(dimethylamino)(heptylmethylamino)methylene]-1,4-piperazinedicarboximidamide (RN: 97963-91-2),
N″,N″″′-[(1,1,3,3-tetramethoxy-1,3-disiloxanediyl)di-3,1propanediyl]bis[N,N,N′,N′-tetramethyl-guanidine] (RN: 69755-28-8),
N″,N″″′-1,6-hexanediylbis[N,N,N′,N′-tetramethyl-guanidine] (RN: 64933-93-3),
N″,N″″′-(methylenedi-4,1-phenylene)bis[N,N,N′,N′-tetramethyl-guanidine](RN: 57414-23-0),
N,N″-(methylphenylene)bis[N′-[bis(dimethylamino)methylene]-urea (RN: 39529-23-2), and
2,2′-(sulphonyldiethylene)bis[1,1,3,3-tetramethyl guanidine] (RN: 13998-89-5).

In another embodiment, the compounds of formula (I) may be chosen from:
N-[bis(dimethylamino)methylene]-3-(dimethylamino)-5-imino-2-methyl-2,4,6,19-tetraazaeicosan-20-imidamide, (RN: 791543-84-5),
N,N′-bis(tetrahydro-1,3-dimethyl-2(1H)-pyrimidinylidene)-1,3-propanediamine (RN: 752232-69-2),
poly(oxy-1,2-ethanediyl), α-[2-[(1,3-dimethyl-2imidazolidinylidene)amino]ethyl]-ω-[2-[(1,3-dimethyl-2imidazolidinylidene)amino]ethoxy] (RN: 742679-23-8),
N″,N″″′-1,2-ethanediylbis[N,N,N′,N′-tetrakis(1-methylethyl)-guanidine (RN: 676488-06-5),
N,N′-bis(1,3-dihydro-1,3-dimethyl-2H-imidazol-2-ylidene)-1,8-naphthalenediamine (RN: 634192-99-7),
N,N′-bis(1,3-dimethyl-2-imidazolidinylidene)-1,8-naphthalenediamine (RN: 501931-38-0),
N″,N″″′-1,2-phenylenebis[N,N,N′,N′-tetramethyl-guanidine] (RN: 495408-35-0),
N″,N″″′-1,8-naphthalenediylbis[N,N,N′,N′-tetramethyl-guanidine]hydrochloride (RN: 443892-20-4),
N″,N″″′,N″″″″-(nitrilotri-2,1-ethanediyl)tris[N,N,N′,N′-tetramethyl-guanidine] (RN: 368866-05-1),
N″,N″″′-[2-[[[bis(dimethylamino)methylene]amino]methyl]-2-methyl-1,3-propanediyl]bis[N,N,N′,N′-tetramethyl-guanidine] (RN: 289474-30-2),
N″,N″″′-(2,2-dimethyl-1,3-propanediyl)bis[N,N,N′,N′-tetramethyl-guanidine] (RN: 289474-29-9),
N″,N″″′-1,3-propanediylbis[N,N,N′,N′-tetramethyl-guanidine] (RN: 289474-28-8),
N,N′-bis(1,3-dihydro-1,3-dimethyl-2H-imidazol-2-ylidene)-1,2-ethanediamine(RN: 216873-26-6),
N,N″′-1,2-ethanediylbis[N′,N′,N″,N″-diethyl-guanidine](RN: 211869-99-7),
N″,N″″′-[N,N,N′,N′-tetramethyl-guanidine] (RN: 196405-86-4),
N″,N″″′-[oxybis(2,1-ethanediyloxy-3,1-propanediyl)]bis[N,N,N′,N′-tetramethyl-guanidine] (RN: 190442-53-6),
N″,N″″′-[[1,1,3,3-tetramethyl-1,3-disiloxanediyl]di-3,1-propanediyl]bis[N,N,N′,N′-tetramethylguanidine] (RN: 175989-14-7),
N,N″-1,6-hexanediylbis[N′-[bis(dimethylamino)methylene urea (RN: 157362-45-3),
N″,N″″′-[N,N,N′,N′-tetramethyl-guanidine] (RN: 144576-63-6),
N,N′-bis(1,3-dimethyl-2-imidazolidinylidene)-1,2-ethanediamine (RN: 126620-51-7),
N″,N″″′-1,4-phenylenebis[N,N,N′,N′-tetramethyl-guanidine] (RN: 113551-45-4),
N″,N″″′-[(1,1,3,3-tetramethoxy-1,3-disiloxanediyl)di-3,1-propanediyl]bis[N,N,N′,N′-tetramethyl-guanidine] (RN: 69755-28-8),
N″,N″″′-1,6-hexanediylbis[N,N,N′,N′-tetramethyl-guanidine] (RN: 64933-93-3), and
2,2′-(sulphonyldiethylene)bis[1,1,3,3-tetramethyl-guanidine] (RN: 13998-89-5).

In the compositions that are intended for shaping keratin fibers, the at least one polyguanidine not belonging to the hydroxide family can be present in the composition in a molar concentration ranging from 0.01 M to 4 M, which corresponds to a concentration ranging from 0.14 wt.% to 160 wt.%, relative to the total weight of the composition, for example, a molar concentration ranging from 0.05 M to 2 M, which corresponds to a concentration ranging from 0.7 wt.% to 80 wt.%, relative to the total weight of the composition.

The pH of the compositions according to the present disclosure may range, for example, from 9.6 to 14, for example, from 11 to 13.

In at least one embodiment, in the compositions of the present disclosure, the at least one polyguanidine not belonging to the hydroxide family can be the only active ingredient for shaping in the composition.

The compositions according to the present disclosure can also comprise at least one reducing agent, for example, thioglycolic acid and/or thiolactic acid and/or their ester and amide derivatives, such as glycerol monothioglycolate; cysteamine and its C₁-C₄ acylated derivatives, such as N-acetyl-cysteamine and N-propionyl-cysteamine; cysteine; N-acetyl-cysteine; thiomalic acid; pantheteine; 2,3-dimercaptosuccinic acid; sulphites and bisulphites of an alkali metal or alkaline-earth metal; N-(mercaptoalkyl)-ω-hydroxyalkylamides, described, for example, in European Patent Application No. 0 354 835; N-mono- or N,N-dialkylmercapto-4-butyramides, described, for example, in European Patent Application No. 0 368 763; aminomercaptoalkylamides, described, for example, in European Patent Application No. 0 432 000; derivatives of N-(mercaptoalkyl)succinamic acids and of the N-(mercaptoalkyl)succinimides, described, for example, in European Patent Application No. 0 465 342; alkylamino mercaptoalkylamides, described, for example, in European Patent Application No. 0 514 282; azeotropic mixtures of 2-hydroxypropyl thioglycolate and of (2-hydroxy-1-methyl)ethyl thioglycolate, described, for example, in French Patent Application No. 2 679 448; mercaptoalkylaminoamides, described, for example, in French Patent Application No. 2 692 481; N-mercaptoalkylalkanediamides, described, for example, in European Patent Application No. 0 653 202, and derivatives of formamidine sulphinic acid, described, for example, in International Patent Application No. PCT/US01/43124.

When the compositions according to the present disclosure comprise at least one reducing agent, the latter may be present in the composition in an amount less than or equal to 20 wt.%, for example, ranging from 0.1 to 10 wt.%, relative to the total weight of the composition.

The compositions according to the present disclosure may also comprise at least one hydroxide chosen, for example, from hydroxides of alkali metals; hydroxides of alkaline-earth metals; hydroxides of transition metals; and organic hydroxides, such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide, and quaternary ammonium hydroxides.

When the compositions of the present disclosure comprise at least one hydroxide, the latter may be present in the composition in an amount ranging from 0.01 wt.% to 3.5 wt.%, for example, from 0.05 to 1.5 wt.%, relative to the total weight of the composition.

According to one embodiment of the present disclosure, the compositions may not contain a base belonging to the hydroxide family, chosen, for example, from the hydroxides of alkali metals, hydroxides of alkaline-earth metals, hydroxides of transition metals, and organic hydroxides.

In at least one embodiment, the compositions of the present disclosure can comprise water in an amount ranging from 0 to 50%, for example, from 0 to 30%, or from 0 to 20%.

According to another embodiment, the basic compositions may also comprise at least one surfactant chosen from non-ionic, anionic, cationic, and amphoteric surfactants, for example, alkylsulphates, alkylbenzenesulphates, alkylethersulphates, alkylsulphonates, quaternary ammonium salts, alkylbetaines, oxyethylenated alkylphenols, alkanolamides of fatty acids, oxyethylenated esters of fatty acids, and non-ionic surfactants of the hydroxypropylether type.

When the basic compositions comprise at least one surfactant, the latter may be present in the composition in an amount less than or equal to 30 wt.%, for example, ranging from 0.5 to 10 wt.%, relative to the total weight of the composition.

The basic composition according to the present disclosure may also comprise at least one treatment agent chosen from cationic, anionic, non-ionic, and amphoteric agents, for the purposes of improving the cosmetic properties of the hair, to tone them down, and/or to prevent their degradation.

Non-limiting examples of treatment agents include those described in French Patent Nos. 2 598 613 and 2 470 596. Additional examples of treatment agents include, but are not limited to, volatile or non-volatile, linear or cyclic silicones and mixtures thereof; polydimethylsiloxanes; quaternized polyorganosiloxanes, such as those described in French Patent Application No. 2 535 730; polyorganosiloxanes with aminoalkyl groups modified by alkoxycarbonyalkyl groups, such as those described in U.S. Pat. No. 4,749,732; polyorganosiloxanes, such as polydimethylsiloxane-polyoxyalkyl copolymers of the Dimethicone Copolyol type; polydimethylsiloxanes with stearoxy terminal groups (stearoxydimethicone); polydimethylsiloxane-dialkylammonium acetate copolymers and polydimethylsiloxanepolyalkylbetaine copolymers described, for example, in British Patent No. 2,197,352; organopolysiloxanes modified by mercapto or mercaptoalkyl groups, such as those described in French Patent No. 1 530 369 and in European Patent Application No. 0 295 780; and silanes, such as stearoxytrimethylsilane.

The basic compositions according to the present disclosure may also comprise at least one other treatment ingredient chosen, for example, from cationic polymers, such as those used in the compositions of French Patent Nos. 2 472 382 and 2 495 931; cationic polymers of the ionene type, such as those used in the compositions of Luxembourg Patent No. 83703; basic amino acids (such as lysine and arginine); acidic amino acids (such as glutamic acid and aspartic acid); peptides and their derivatives; hydrolysates of proteins; waxes; swelling agents; penetrating agents; agents for reinforcing the effectiveness of the reducing agent, such as the SiO₂/PDMS (polydimethylsiloxane) mixture; dimethylisosorbitol; urea and its derivatives; pyrrolidone; N-alkylpyrrolidones; thiamorpholinone; alkyleneglycol and dialkyleneglycol alkyl ethers, such as propyleneglycol monomethyl ether, dipropyleneglycol monomethyl ether, ethyleneglycol monoethyl ether, and diethyleneglycol monoethylether; imidazolidinone-2; fatty alcohols; derivatives of lanolin; active ingredients, such as pantothenic acid; agents for preventing hair loss; anti-dandruff agents; thickeners; suspending agents; sequestering or complexing agents; opacifying agents; sun filters; perfumes; and preservatives.

In at least one embodiment of the present disclosure, the at least one polyguanidine not belonging to the hydroxide family may shape keratin fibers without being brought into contact with an organic solvent beforehand.

The compositions according to the present disclosure may, in at least one embodiment, be presented in the form of a thick cream, so as to keep the hair as stiff as possible. These creams may be in the form of “heavy” emulsions, for example, based on glyceryl stearate, glycol stearate, auto-emulsifiable waxes, and/or fatty alcohols.

It is also possible to use liquids or gels containing thickening agents, such as carboxyvinyl polymers and copolymers, which “glue” the hair and maintain it in a smooth position during the holding time.

The compositions according to the present disclosure may additionally comprise at least one additive chosen from silicones (in soluble, dispersed, and/or micro-dispersed form); non-ionic, anionic, cationic, and amphoteric surfactants; ceramides; glycoceramides; pseudo-ceramides; vitamins; pro-vitamins, including panthenol; vegetable, animal, mineral, and synthetic oils; waxes other than the ceramides, glycoceramides, and pseudo-ceramides; water-soluble and fat-soluble, siliconized or non-siliconized sun filters; lustre agents; opacifiers; sequestering agents; plasticizers; solubilizers; acidifiers; organic and inorganic thickening agents; antioxidants; hydroxyacids; penetrating agents; perfumes; and preservatives. Examples of suitable solubilizers include, but are not limited to, lower alcohols, such as ethanol, propanol, and isopropanol.

Also disclosed herein is a kit comprising at least two compartments, at least one compartment (i) comprising, in a cosmetically acceptable medium, at least one polyguanidine not belonging to the hydroxide family, wherein said at least one polyguanidine can react on the cystines of the keratin fibers, via a beta-elimination reaction, to produce dehydroalanine and leading to the formation of lanthionine, to form curls in the keratin fibers with a diameter ranging from 0.2 to 3 cm, in a period of time of less than 60 minutes.

In at least one embodiment, the kit according to the present disclosure may additionally comprise at least one supplementary composition (ii) for care, conditioning, make-up, make-up removal, protection, cleaning, and/or washing of keratin fibers.

The compositions of the kits according to the present disclosure may be packaged in separate compartments or containers or devices, optionally accompanied by appropriate, identical or different, applicators, such as brushes, sponges, etc.

Also disclosed herein is a method of shaping keratinous materials comprising applying a cosmetic composition to the keratinous materials, wherein the cosmetic composition comprises, in a cosmetically acceptable medium, at least one polyguanidine not belonging to the hydroxide family, the cosmetically acceptable medium and the at least one polyguanidine being chosen such that the at least one polyguanidine not belonging to the hydroxide family reacts on the cystines of the keratinous materialss, by a beta-elimination reaction, producing dehydroalanine and leading to the formation of lanthionine, to form curls in the keratinous materials with a diameter ranging from 0.2 to 3 cm, wherein the method occurs in a period of time of less than 60 minutes.

In at least one embodiment of the present disclosure, the period of time for shaping may be less than or equal to 40 minutes, for example, less than or equal to 30 minutes.

Further disclosed herein is a method of permanent shaping of keratinous materials comprising applying a cosmetic composition to the keratinous materials, wherein the cosmetic composition comprises, in a cosmetically acceptable medium, at least one imine not belonging to the hydroxide family, the cosmetically acceptable medium, and wherein the imine is chosen such that the imine not belonging to the hydroxide family reacts on the cystines of the keratin fibers, by a beta-elimination reaction, producing dehydroalanine and leading to the formation of lanthionine, to form curls in the keratin fibers with a diameter ranging from 0.2 to 3 cm, wherein the method occurs in a period of time of less than 60 minutes.

In at least one embodiment, the period of time for permanent shaping may be less than or equal to 40 minutes, for example, less than or equal to 30 minutes.

In the method of permanent shaping according to the present disclosure, the composition can be applied on dry or previously wetted hair, previously rolled up on rollers or curlers, having a diameter ranging from 2 to 30 mm, it being possible for the composition to be applied as the hair is rolled up; the composition can then be left to act for a period of time ranging from 5 to 60 minutes, for example, from 5 to 40 minutes. After removing the rollers, the hair can be rinsed copiously.

It is possible to use a heating iron both as heating means and hair smoothing means, at a temperature ranging, for example, from 60 to 220° C., for instance, from 120 to 200° C.

Also disclosed herein is an active agent for shaping keratin fibers, by means of a beta-elimination reaction, producing dehydroalanine and leading to the formation of lanthionine, comprising at least one polyguanidine not belonging to the hydroxide family.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Example 1

A simplified perming composition was prepared containing N″,N″″′-1 ,3-propanediylbis[N,N,N′,N′-tetramethyl-guanidine] (RN: 289474-28-8) at a concentration of 0.5 M in water, as the perming agent. This composition was applied to Caucasian hair, previously rolled up on curlers, for 15 minutes at a temperature of 30° C. The hair was rinsed and dried. The hair had beautiful curls and was soft to the touch.

### Example 2

A simplified perming composition was prepared containing N″,N″″′,N″″″″-(nitrilotri-2,1-ethanediyl)tris[N,N,N′,N′-tetramethyl-guanidine] (RN: 368866-05-1) at a concentration of 0.25 M in water, as the perming agent. This composition was applied to Caucasian hair, previously rolled up on curlers, for 25 minutes at a temperature of 30° C. The hair was rinsed and dried. The hair had beautiful curls and was soft to the touch.

What is claimed is:

1. A cosmetic composition for shaping keratin fibers comprising, in a cosmetically acceptable medium, at least one polyguanidine not belonging to the hydroxide family, the cosmetically acceptable medium, and the at least one polyguanidine being chosen such that the at least one polyguanidine not belonging to the hydroxide family reacts on the cystines of the keratin fibers, by a beta-elimination reaction, producing dehydroalanine and leading to the formation of lanthionine, to form curls in the keratin fibers with a diameter ranging from 0.2 to 3 cm, in a period of shaping time of less than 60 minutes.

2. The cosmetic composition of claim 1, wherein the period of shaping time is less than or equal to 40 minutes.

3. The cosmetic composition of claim 2, wherein the period of shaping time is less than or equal to 30 minutes.

4. The cosmetic composition of claim 1, wherein the at least one polyguanidine not belonging to the hydroxide family is present in the composition in a molar concentration ranging from 0.01 M to 4 M.

5. The cosmetic composition of claim 4, wherein the at least one polyguanidine not belonging to the hydroxide family is present in the composition in a molar concentration ranging from 0.05 M to 2 M.

6. The cosmetic composition of claim 1, wherein the pH of the composition ranges from 9.6 to 14.

7. The cosmetic composition of claim 6, wherein the pH of the composition ranges from 11 to 13.

8. The cosmetic composition of claim 1, wherein the composition does not comprise a base belonging to the hydroxide family.

9. The cosmetic composition of claim 1, optionally further comprising water in an amount ranging from 0 and 50%.

10. The cosmetic composition of claim 9, wherein water is present in an amount ranging from 0 to 30%.

11. The cosmetic composition of claim 10, wherein water is present in an amount ranging from 0 and 20%.

12. The composition of claim 1, wherein the at least one polyguanidine is chosen from compounds of formula (I): 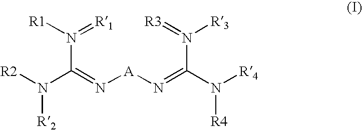
wherein:
R₁, R′₁, R₂, R′₂, R₃, R′₃, R₄, and R′₄, which may be identical or different, are radicals chosen from:
hydrogen; and
C₁-C₆ alkyl groups, which may be saturated or unsaturated, linear, branched, or cyclic, said alkyl radicals being optionally substituted by a radical of formula (II): 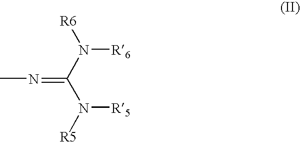
wherein R₅, R′₅, R₆, and R′₆, which may be identical or different, are radicals chosen from:
hydrogen; and
C₁-C₆ alkyl groups, which may be saturated or unsaturated, linear, branched, or cyclic,
R₁ and R₂ and/or R₃ and R₄ may optionally form together a divalent radical chosen from (CH₂)₂, (CH₂)₃, and CH═CH,
A is chosen from C₂-C₁₂ divalent hydrocarbon radicals, which may be linear or cyclic, saturated or unsaturated, and which may optionally be interrupted by at least one entity chosen from:
imino, carboxamido, sulphoxide, and sulphone groups, and/or
at least one heteroatom chosen from sulphur, oxygen, nitrogen, and silicon, and optionally substituted by:
a linear or branched C₁-C₄ alkyl radical, optionally interrupted by at least one heteroatom chosen from sulphur, oxygen, nitrogen, and silicon, and optionally substituted by a radical of formula (II).

13. The cosmetic composition of claim 12, wherein at least one of the C₁-C₆ alkyl groups of R₁, R′₁, R₂, R′₂, R₃, R′₃, R₄, and R′₄, R₅, R′₅, R₆, and R′₆ is a linear group chosen from methyl and ethyl groups.

14. The cosmetic composition of claim 12, wherein at least one of the C₁-C₆ alkyl groups of R₁, R′₁, R₂, R′₂, R₃, R′₃, R₄, and R′₄, R₅, R′₅, R₆, and R′₆ is a branched group chosen from isopropyl and tert-butyl groups.

15. The cosmetic composition of claim 12, wherein at least one of the C₁-C₆ alkyl groups of R₁, R′₁, R₂, R′₂, R₃, R′₃, R₄, and R′₄, R₅, R′₅, R₆, and R′₆ is a cyclic group chosen from cyclopentyl and cyclohexyl groups.

16. The cosmetic composition of claim 1, wherein the compounds of formula (I) are chosen from:
N,N′-bis(1,3-dimethyl-2-imidazolidinylidene)-1,2-benzenediamine (RN: 774610-65-0),
N-[bis(dimethylamino)methylene]-3-(dimethylamino)-5-imino-2-methyl-2,4,6,19-tetraazaeicosan-20-imidamide (RN: 791543-84-5),
N,N′-bis(tetrahydro-1,3-dimethyl-2(1H)-pyrimidinylidene)-1,3-propanediamine (RN: 752232-69-2),
1,1′,1″,1″′-[1-propanediylbis(nitrilomethanetetrayl)]tetrakis-piperidine (RN: 752232-68-1),
poly(oxy-1,2-ethanediyl), α-[2-[(1,3-dimethyl-2-imidazolidinylidene)amino]ethyl]-ω-[2-[(1,3-dimethyl-2imidazolidinylidene)amino]ethoxy] (RN: 742679-23-8),
N″,N″″′-1,2-ethanediylbis[N,N,N′,N′-tetrakis(1-methylethyl)-guanidine (RN: 676488-06-5)
N,N′-bis(1,3-dihydro-1,3-dimethyl-2H-imidazol-2-ylidene)-1,8-naphthalenediamine (RN: 634192-99-7),
N,N′-bis(1,3-dimethyl-2-imidazolidinylidene)-1,8-naphthalenediamine (RN: 501931-38-0),
N″,N″″′-1,8-biphenylenediylbis[N,N,N′,N′-tetramethyl-guanidine] (RN: 495408-66-7),
N″,N″″′-benzo[c]phenanthrene-1,12-diylbis[N,N,N′,N′tetramethyl-guanidine] (RN: 495408-58-7),
N″,N″″′-4,5-phenanthrenediylbis[N,N,N′N′-tetramethyl-guanidine] (RN: 495408-50-9),
N″,N″″′-(9,10-dihydro-4,5-phenanthrenediyl)bis[N,N,N′N′-tetramethyl-guanidine] (RN: 495408-44-1),
N″,N″″′-1,2-phenylenebis[N,N,N′N′-tetramethyl-guanidine] (RN: 495408-35-0),
N″,N″″′-1,8-naphthalenediylbis[N,N,N′N′-tetramethyl-guanidine] hydrochloride (RN: 443892-20-4),
N″,N″″′-9H-fluorene-4,5-diylbis[N,N,N′N′-tetramethyl-guanidine] (RN: 443892-12-4),
N′-(tetrahydro-1,3-dimethyl-2(1H)-pyrimidinylidene)-N,N-bis[2-[(tetrahydro-1,3-dimethyl-2(1H)-pyrimidinylidene)amino]ethyl]-1,2-ethanediamine (RN: 395640-62-7),
N″,N″″′,N″″″″-(nitrilotri-2,1-ethanediyl)tris[N,N,N′N′-tetramethyl-guanidine] (RN: 368866-05-1),
N″,N″″′-[2-[[[bis(dimethylamino)methylene]amino]methyl]-2-methyl-1,3-propanediyl]bis[N,N,N′N′-tetramethyl-guanidine] (RN: 289474-30-2),
N″,N″″′-(2,2-dimethyl-1,3-propanediyl)bis[N,N,N′N′-tetramethyl-guanidine] (RN: 289474-29-9),
N″,N″″′-1,3-propanediylbis[N,N,N′,N′-tetramethyl-guanidine](RN:289474-28-8),
N″,N″″′-1,2-ethanediylbis[N,N,N′N′-tetramethyl-guanidine] (RN: 289474-25-5),
N,N′-bis(1,3-dihydro-1,3-dimethyl-2H-imidazol-2-ylidene)-1,2-ethanediamine (RN: 216873-26-6),
N,N″′-1,2-ethanediylbis[N′,N′,N″,N″-diethyl-guanidine] (RN: 211869-99-7),
N″,N″″′-[(methylimino)di-3,1-propanediyl]bis[N,N,N′N′-tetramethyl-guanidine] (RN: 196405-864),
N″,N″″′-[oxybis(2,1-ethanediyloxy-3,1-propanediyl)]bis[N,N,N′,N′-tetramethyl-guanidine] (RN: 190442-53-6),
N″,N″″′-[[1,1,3,3-tetramethyl-1,3-disiloxanediyl]di-3,1propanediyl]bis[N,N,N′,N′-tetramethylguanidine] (RN: 175989-14-7),
N″,N″″′-[[1,3-dimethyl-1,3-bis[(trimethylsilyl)oxy]-1,3-disiloxanediyl]di-3,1-propanediyl]bis[N,N,N′N′-tetramethyl-guanidine] (RN: 172283-48-6),
N,N″-1,6-hexanediylbis[N′-[bis(dimethylamino)methylene urea (RN: 157362-45-3),
[bis(dimethylamino)methylene][3[[[[[bis(dimethylamino)-methylene]amino]carbonyl]amino]methyl]-3,5,5-trimethylcyclohexyl]-urea (RN: 157362-44-2),
N″,N″″′-[1,1′-biphenyl]-2,2′-diylbis[N,N,N′N′-tetramethyl-guanidine] (RN: 144576-63-6),
4,4′-sulphonylbis[N-(1,3-dimethyl-2-imidazolidinylidene)-benzeneamine (RN: 129346-76-5),
N,N′-bis(1,3-dimethyl-2-imidazolidinylidene),2,8-dibenzothiophenediamine-5,5-dioxide (RN: 128169-35-7),
N,N′-bis(1,3-dimethyl-2-imidazolidinylidene)-4,6-dibenzothiophenediamine-5,5-dioxide (RN: 127330-56-7),
N,N′-bis(1,3-dimethyl-2-imidazolidinylidene)-1,2-ethanediamine (RN: 126620-51-7),
N″,N″″′-1,3-phenylenebis[N,N,N′N′-tetramethyl-guanidine] (RN: 121648-84-8),
N,N′-bis[bis(dimethylamino)methylene]-butanediamine (RN: 114491-72-4),
N″,N″″′-1,4-phenylenebis[N,N,N′N′-tetramethyl-guanidine] (RN: 113551-45-4),
N″,N″″′-[[1,3-dimethyl-1-[(pentamethyidisiloxanyl)oxy]-3-[(trimethylsilyl)oxy]-1,3-disiloxanediyl]di-3,1-propanediyl]bis[N,N,N′,N′-tetramethyl-guanidine] (RN: 109956-31-2),
N,N″-bis[(dimethylamino)(dipentylamino)methylene]-1,4piperazinedicarboximidamide (RN: 97983-93-2),
N,N″-bis[(dimethylamino)(hexylpropylamino)methylene]-1,4piperazinedicarboximidamide (RN: 97983-92-1),
N,N″-bis[(hexylmethylamino)(methylpropylamino)methylene]-1,4-piperazinedicarboximidamide (RN: 97983-91-0),
N,N″-bis[(butylmethylamino)(hexylmethylamino)methylene]-1,4-piperazinedicarboximidamide (RN: 97983-90-9),
N,N″-bis[(dimethylamino)(heptylmethylamino)methylene]-1,4-piperazinedicarboximidamide (RN: 97963-91-2),
N″,N″″′-[(1,1,3,3-tetramethoxy-1,3-disiloxanediyl)di-3,1propanediyl]bis[N,N,N′,N′-tetramethyl-guanidine] (RN: 69755-28-8),
N″,N″″′-1,6-hexanediylbis[N,N,N′N′-tetramethyl-guanidine] (RN: 64933-93-3),
N″,N″″′-(methylenedi-4,1-phenylene)bis[N,N,N′N′-tetramethyl-guanidine] (RN: 57414-23-0),
N,N″-(methylphenylene)bis[N′-[bis(dimethylamino)methylene]-urea (RN: 39529-23-2), and
2,2′-(sulphonyldiethylene)bis[1,1,3,3-tetramethyl-guanidine] (RN: 13998-89-5).

17. The cosmetic composition of claim 12, wherein the compounds of formula (I) are chosen from:
N-[bis(dimethylamino)methylene]-3-(dimethylamino)-5-imino-2-methyl-2,4,6,19-tetraazaeicosan-20-imidamide, (RN: 791543-84-5),
N,N′-bis(tetrahydro-1,3-dimethyl-2(1H)-pyrimidinylidene)-1,3-propanediamine (RN: 752232-69-2),
poly(oxy-1,2-ethanediyl), α-[2-[(1,3-dimethyl-2imidazolidinylidene)amino]ethyl]-ω-[2-[(1,3-dimethyl-2imidazolidinylidene)amino]ethoxy] (RN: 742679-23-8),
N″,N″″′-1,2-ethanediylbis[N,N,N′,N′-tetrakis(1-methylethyl)-guanidine (RN: 676488-06-5),
N,N′-bis(1,3-dihydro-1,3-dimethyl-2H-imidazol-2-ylidene)-1,8-naphthalenediamine (RN: 634192-99-7),
N,N′-bis(1,3-dimethyl-2-imidazolidinylidene)-1,8-naphthalenediamine (RN: 501931-38-0)
N″,N″″′-1,2-phenylenebis[N,N,N′N′-tetramethyl-guanidine] (RN: 495408-35-0),
N″,N″″′-1,8-naphthalenediylbis[N,N,N′N′-tetramethyl-guanidine]hydrochloride (RN: 443892-20-4),
N″,N″″′,N″″″″-(nitrilotri-2,1-ethanediyl)tris[N,N,N′N′-tetramethyl-guanidine] (RN: 368866-05-1),
N″,N″″′-[2-[[[bis(dimethylamino)methylene]amino]methyl]-2-methyl-1,3-propanediyl]bis[N,N,N′N′-tetramethyl-guanidine] (RN: 289474-30-2),
N″,N″″′-(2,2-dimethyl-1,3-propanediyl)bis[N,N,N′N′-tetramethyl-guanidine] (RN: 289474-29-9),
N″,N″″′-1,3-propanediylbis[N,N,N′N′-tetramethyl-guanidine] (RN: 289474-28-8),
N,N′-bis(1,3-dihydro-1,3-dimethyl-2H-imidazol-2-ylidene)-1,2-ethanediamine (RN: 216873-26-6),
N,N″′-1,2-ethanediylbis[N′,N′,N″,N″-diethyl-guanidine](RN: 211869-99-7),
N″,N″″′-[(methylimino)di-3,1-propanediyl]bis[N,N,N′N′-tetramethyl-guanidine] (RN: 196405-86-4),
N″,N″″′-[oxybis(2,1-ethanediyloxy-3,1-propanediyl)]bis[N,N,N′,N′-tetramethyl-guanidine] (RN: 190442-53-6),
N″,N″″′-[[1,1,3,3-tetramethyl-1,3-disiloxanediyl]di-3,1-propanediyl]bis[N,N,N′,N′-tetramethylguanidine] (RN: 175989-14-7),
N,N″-1,6-hexanediylbis[N′-[bis(dimethylamino)methylene urea (RN: 157362-45-3),
N″,N″″′-[1,1′-biphenyl]-2,2′-diylbis[N,N,N′N′-tetramethyl-guanidine] (RN: 144576-63-6),
N,N′-bis(1,3-dimethyl-2-imidazolidinylidene)-1,2-ethanediamine (RN: 126620-51-7),
N″,N″″′-1,4-phenylenebis[N,N,N′N′-tetramethyl-guanidine] (RN: 113551-45-4),
N″,N″″′-[(1,1,3,3-tetramethoxy-1,3-disiloxanediyl)di-3,1-propanediyl]bis[N,N,N′,N′-tetramethyl-guanidine] (RN: 69755-28-8),
N″,N″″′-1,6-hexanediylbis[N,N,N′N′-tetramethyl-guanidine] (RN: 64933-93-3), and
2,2′-(sulphonyidiethylene)bis[1,1,3,3-tetramethyl guanidine] (RN: 13998-89-5).

18. The cosmetic composition of claim 1, further comprising at least one additive chosen from silicones in soluble, dispersed, or micro-dispersed form; non-ionic, anionic, cationic, and amphoteric surfactants; ceramides; glycoceramides; pseudo-ceramides; vitamins; pro-vitamins; vegetable, animal, mineral, and synthetic oils; waxes other than ceramides, glycoceramides, and pseudo-ceramides; water-soluble and fat-soluble, siliconized or non-siliconized sun filters; lustre agents; opacifiers; sequestering agents; plasticizers; solubilizers; acidifiers; organic and inorganic thickening agents; antioxidants; hydroxyacids; penetrating agents; perfumes; and preservatives.

19. A kit comprising at least two compartments, wherein at least one of the compartments (i) comprises, in a cosmetically acceptable medium, a composition for shaping keratin fibers comprising at least one polyguanidine not belonging to the hydroxide family, capable of reacting on the cystines of the keratin fibers, by a beta-elimination reaction, producing dehydroalanine and leading to the formation of lanthionine, to form curls in keratin fibers with a diameter ranging from 0.2 to 3 cm, in a period of shaping time of less than 60 minutes.

20. The kit of claim 19, further comprising at least one supplementary composition (ii) for care, conditioning, make-up, make-up removal, protection, cleaning, and/or washing of keratin fibers.

21. A method of shaping keratinous materials comprising applying a cosmetic composition to the keratinous materials, wherein the cosmetic composition comprises, in a cosmetically acceptable medium, at least one polyguanidine not belonging to the hydroxide family, the cosmetically acceptable medium and the at least one polyguanidine being chosen such that the at least one polyguanidine not belonging to the hydroxide family reacts on the cystines of the keratinous materials, by a beta-elimination reaction, producing dehydroalanine and leading to the formation of lanthionine, to form curls in the keratinous materials with a diameter ranging from 0.2 and 3 cm, in a period of shaping time of less than 60 minutes.

22. The method of claim 21, wherein after application of the composition, the keratinous materials undergo a thermal treatment by heating to a temperature ranging from 30 to 60° C.

23. The method of claim 21, wherein the keratinous materials are exposed to a heating iron, which is used both as heating means and smoothing means, at a temperature ranging from 60 and 220° C.

24. The method of claim 23, wherein the temperature ranges from 120 to 200° C.

25. The method of claim 13, wherein the period of shaping time is less than or equal to 40 minutes.

26. The method of claim 25, wherein the period of shaping time is less than or equal to 30 minutes.

27. An active agent for shaping keratin fibers, via a beta-elimination reaction, producing dehydroalanine and leading to the formation of lanthionine, comprising at least one polyguanidine not belonging to the hydroxide family.
